(19) United States
(12) Patent Application Publication
(10) Pub. No.: US 2024/0016854 A1
(43) Pub. Date: Jan. 18, 2024

(54) A METHOD OF SNAKE FISH EXTRACTION

(71) Applicant: Sucipto KOKADIR, Jarkata Barat (ID)

(72) Inventor: Sucipto KOKADIR, Jarkata Barat (ID)

(21) Appl. No.: 17/771,419

(22) PCT Filed: Sep. 16, 2020
(86) PCT No.: PCT/IB2020/058587

§ 371 Date: Apr. 22, 2022

(30) Foreign Application Priority Data
Sep. 17, 2019 (ID) P00201908169
Oct. 5, 2019 (ID) P00201903952
Feb. 14, 2020 (ID) S00202001315
Mar. 30, 2020 (ID) S00202002432

(51) Int. Cl. A61K 35/60; A61K 36/185; A61K 47/36; A61K 36/9066

(52) U.S. Cl. CPC A61K 35/60; A61K 36/185; A61K 47/36; A61K 36/9066

(57) ABSTRACT

A method of producing a bioextract from a snake fish comprising steps of selecting of a snake fish, cleaning the snake fish, reducing the snake fish down to manageable pieces by cutting, milling the snake fish, utilizing an extracting process of the milled pulp, utilizing a drying process of the extract filtrate with a combination of maltodextrin DE 10-12, powering the solidified bioextract after drying, and applying the filtered bioproduct to an industrial use. An extraction process of a snake fish may be described which may include the steps of combining a snake fish pulp with purified water and ethanol, having a concentration of about 96%, to make an extraction medium and placing the extraction medium into an extraction machine and waiting about 24 hours. Additionally described herein may be various formulations of a powered bioextract from snake fish, Moringa extract, and/or Curcuma extract.

### RELATED APPLICATIONS

This application claims the benefit of Indonesian patent number P00201908169, filed Sep. 17, 2019; Indonesian patent number S00202001315, filed Feb. 14, 2020; Indonesian patent number P00201903952, filed, Oct. 5, 2019, and Indonesian patent number S00202002432, filed Mar. 30, 2020; the contents of each of which are incorporated by this reference in their entireties for all purposes as if fully set forth herein.

### TECHNICAL FIELD

The disclosure herein relates generally to the extraction process of various species of snake fish. More particularly, this disclosure herein relates to an initial preparation process of snake fish, an extraction process of snake fish, and additional processing for the production of powder for an ingredient for use in supplements.

### BACKGROUND

Snake fish, by way of example, the species Channa striata, may be capable of providing an abundant protein source contained within the fish. Said protein content may not only a source of nutrition but may also hold several benefits for health. One of the fish that may be used as the ingredient for supplement products may be the aforementioned snake fish, including the species Channa striata.

Snake fish may be a tropical freshwater fish with high protein content, especially albumin. Snake fish is believed to be a predator fish that may also go by the name common snakehead, chevron snakehead, snakehead murrel, or may generally be referred to as a mudfish.

The snake fish can grow up to a meter in length, though, this size is rarely found in the wild. It has a wide range covering southern China, Pakistan, most of India, southern Nepal, Bangladesh, Sri Lanka, and most of Southeast Asia. It has more recently been introduced to the outermost parts of Indonesia and can be found on almost every river in Indonesia, the Philippines, and Mauritius.

The snake fish may be an important food fish in its entire native range, and may be of considerable economic importance to ranges outside the native habitat. Adults may be dark brown with faint black bands visible across its entire body. Males and females both help to construct a nest out of water vegetation during breeding time. Eggs may be guarded by both parents. Fry may be reddish-orange and may be guarded by both parents until they turn greenish-brown at around 5-6 cm.

It is common in freshwater plains, where it migrates from rivers and lakes into flooded fields, returning to the permanent water bodies in the dry season, where it survives by burrowing in the mud. It preys on frogs, water bugs, and smaller fish, and it may attack anything moving when breeding.

What is needed is an extraction process that makes better use and utilization of the snake fish. Because the snake fish has a nutritional content that is complete, and therefore, beneficial for bodily functions, an extraction process is needed to thoroughly obtain its maximum benefits. Accordingly, such processing of the snake fish to produce an extract powder may confer benefits such as simplifying the consumption method, thereby making it easy for anyone to consume it. Such simplicity in consumption may enable individuals who don't like snake fish to consume the extract thereby providing said nutritional benefits without any flavoring.

### SUMMARY

Certain deficiencies of the prior art are overcome by the provision of embodiments of an apparatus, kit, and system in accordance with the present disclosure. Described herein may be a method of producing a bioextract from a snake fish comprising the steps of a selecting of a snake fish, cleaning the snake fish, reducing the snake fish down to manageable pieces by cutting, milling of the snake fish, utilizing an extracting process of the milled pulp, utilizing a drying process of the extract filtrate with a combination of maltodextrin DE 10-12 prior to the drying process, powering the solidified bioextract after drying, and, the applying the filtered bioproduct to a relevant industrial use.

Additionally described herein may be an extraction process of a snake fish which may include the steps of combining a snake fish pulp with purified water and ethanol, having a concentration of about 96%, to make an extraction medium. This may be followed by placing the extraction medium into an extraction machine and waiting for the extraction of the extraction medium to occur over about 24 hours. It may be important to utilize a temperature control setting for the extraction process so that the extraction medium is extracted at a temperature range of about 50 degrees centigrade to about 65 degrees centigrade. The extraction process may be complete when the 96% ethanol has evaporated leaving behind an extracted medium. A filtration step of the extracted medium may be needed wherein a filter paper is used to separate a filtrate from a residue. The filtrate may be about 40% to 50% of the extracted medium. Then a drying process of the filtrate to make a solid bioextract may be necessary. The drying process may leave a solid cake type material and may need to be milled to make a powered bioextract.

Additionally described herein may be various formulations of a powered bioextract from a snake fish, with a Moringa extract, and with a Curcuma extract. Additionally, the snake fish extract may be used alone, or in combination with only the Moringa extract or only the Curcuma extract.

### BRIEF DESCRIPTION OF THE DRAWINGS

The foregoing and other features of the present disclosure may become more fully apparent from the following description, taken in conjunction with the accompanying drawings. Understanding that these drawings depict only several embodiments in accordance with the disclosure and are not to be considered limiting of its scope, the disclosure may be described with additional specificity and detail through the use of accompanying drawings. Accordingly, further advantages of the present disclosure may become apparent to those skilled in the art with the benefit of the following detailed description of the preferred embodiments and upon reference to the accompanying drawings in which:

FIG. 1 is a flow chart describing one non-limiting method disclosed herein; and,

FIG. 2 is a flow chart describing one non-limiting process disclosed herein.

### DETAILED DESCRIPTION OF THE PREFERRED EMBODIMENTS

Embodiments of systems, components, and methods of assembly and manufacture may now be described with reference to the accompanying figures. Although several embodiments, examples, and illustrations are disclosed below, it may be understood by those of ordinary skill in the art that the embodiments described herein extend beyond the specifically disclosed configurations, examples, and illustrations, and can include other users of the disclosure and obvious modifications and equivalents thereof. The terminology used in the descriptions presented herein is not intended to be interpreted in any limited or restrictive manner simply because it is being used in conjunction with a detailed description of certain specific embodiments of the disclosure. In addition, embodiments of the disclosure can comprise several novel features and no single feature is solely responsible for its desirable attributes or is essential to practicing any one of the several embodiments herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” “top,” “bottom” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specially mentioned above, derivatives thereof, and words of similar import. Further, the use of the term “about” as used herein may be defined as approximately the value as stated.

Referring to the drawings, like reference numerals designate identical or corresponding features throughout the several views. Described herein are certain non-limiting embodiments of an extraction process and method for snake fish. Accordingly, the high albumin content within the snake fish makes this fish ideal as a consumption product by many people to fix hypoalbuminemia conditions.

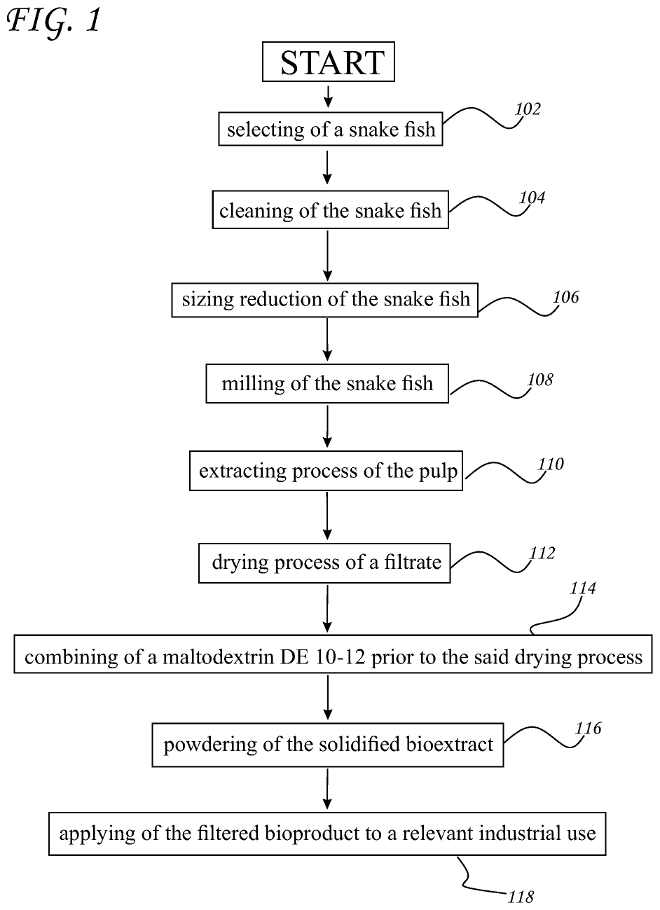

Referring to FIG. 1, said extraction process may be generally and broadly described through the following steps; fish selection 102, cleaning 104, size reduction 106, milling 108, extraction process 110, drying 112, adding maltodextrin DE 10-12 114 (this step may occur before or after the drying step), powdering 116, and product application 118. While the reference numbers have been indicated in this paragraph, it should be understood that like, or similar, steps described herein may carry the same reference number even though not explicitly applied.

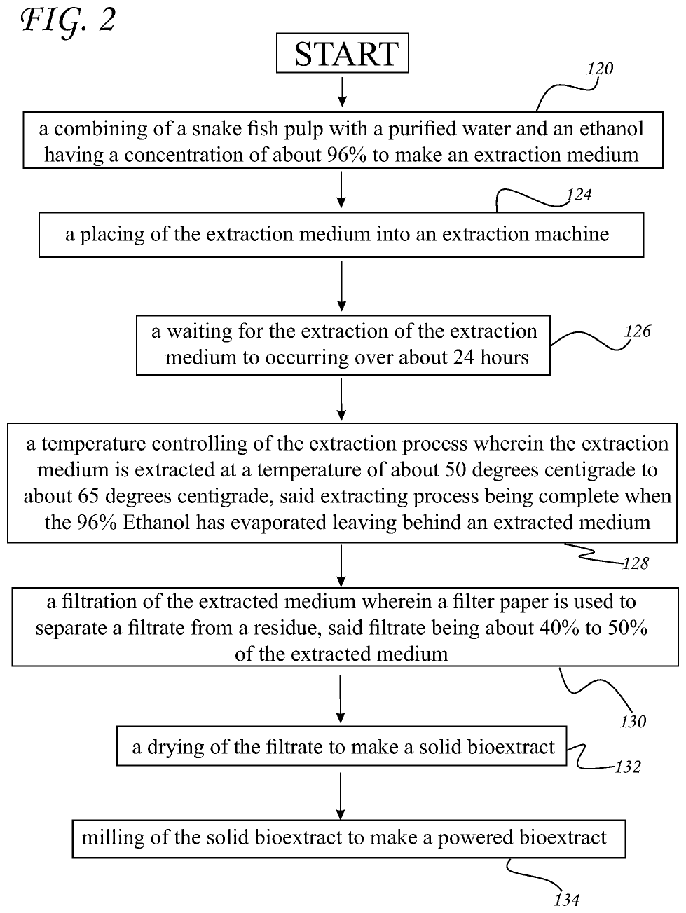

Referring to FIG. 2, an extraction process may generally and broadly proceed by the indicated reference numbers of combining 120, placing 124, waiting 126, temperature controlling 128, filtering the extraction 130, drying the filtrate 132, and milling 134. While the reference numbers have been indicated in this paragraph, it should be understood that like, or similar, steps described herein may carry the same reference number even though not explicitly applied.

The fish selection process as mentioned may be performed by selecting the fish with a mass of 250 grams or greater. This ideal specification may have a high protein content and low in fat. The cleaning process as mentioned may be performed by removing the scales and guts of the fish, followed by the rinsing or washing using a flow of water. The size reduction process as mentioned may be performed by cutting the fish from tail to head into 3 to 5 pieces. The milling process as mentioned may be performed on the whole part of the fish including the skin, bones, and head that has been cleaned. The milling may be performed using a milling machine until a fine pulp of fish is obtained. The extraction process as mentioned may be performed using a solvent, which may be a combination of purified water and ethanol at a concentration of about 96%. The concentration of fish pulp, ethanol, and purified water may be approximately 50%-70%, 2%-10%, and 25%-50%, respectively. The extraction process may be performed at a temperature of approximately degrees centigrade to approximately 70 degrees centigrade, with an extraction time of approximately 12-24 hours. The drying process as mentioned may be performed with an addition of maltodextrin DE 10-12 with approximately 20%-60% concentration. The drying process may be performed by drying the solution using an oven with a temperature range of approximately 50 degrees centigrade to 90 degrees centigrade.

The dried extract may be white with a little trace yellow to brown, and a faintly fishy smell. The dried extract may then go through a powdering process as mentioned by milling the solidified extract until a powder with an approximate size of 65-100% mesh is obtained.

The extract powder produced may be applied as one out of several ingredients for supplement products. The supplement may be packaged in the form of a capsule, liquid sachet, or powder sachet. The capsule supplement may have a total mass of 500 mg, with 100% snake fish extract content. The powder sachet supplement may have a gr of total mass, where the fish extract occupies 50% of the total extract, and the other 50% may be the combination of Moringa and Curcuma extract. The liquid sachet supplement may be 15 ml consisting of snake fish, Moringa, and Curcuma extract. The snake fish extract in this product may comprise only 5% in concentration. This 5% may also be a combination of snake fish and Moringa extract, snake fish and Curcuma extract, or snake fish, Moringa, and Curcuma extract. This product is the first in both Indonesia and the world. The snake fish extract powder produced as mentioned may serve to increase the albumin content within the body, so hypoalbuminemia condition can be corrected or prevented.

The extraction of snake fish is usually done using the flesh by boiling it and then dry the boiled water for extraction. This method leaves all other beneficial parts such as the skin, bone, and head to waste. This disclosure herein may design an extraction method where all beneficial parts of the fish may be used for extraction. The extraction process may be performed with the use of a polar solvent which may be the combination of water (or de-ionized water) and ethanol. The time and temperature of extraction may be set using a standardized value to obtain a maximum extract quantity with high nutritional contents. This extraction process's main concern may be in the albumin content, while the other additional concerns may be the content of protein, amino acid, and mineral.

The production process may be started by selecting the fish with a total mass of >250 grams per fish. The fish may then be processed hygienically in such a way that the main active and beneficial contents of the fish may be still intact. The extracted liquid may then be dried and milled to produce an extract powder. This extract powder may then be applied as the ingredients for various medical supplements.

The supplement that may be made using the snake fish extract powder as one of its supplements may be in the form of a capsule, liquid sachet, or powder sachet. This product may be commercialized and may be a supplement product with snake fish extract as the main ingredient.

This disclosure may be a further development of the previous patent with patent number P0020183943 (herein incorporated by reference), registered on 7 Dec. 2018, by an LPPM organization of Hasanuddin University in Makassar about the production of snake fish protein nano concentrate using an ultrasonic homogenization method. Another patent with a patent number of S00201701034 (herein incorporated by reference) registered on 19 May 2017 by Prof. Dr. Nurpudji Astuti, MPH about high protein products made from snake fish.

Based on a review of the above two patents, the extraction of snake fish is customarily only done by boiling the fish, followed by drying the boiled water to make a powder extract. This disclosure herein may describe the use of a solvent during the extraction process, a method that has yet to be done on the previous patents.

Patent number IDP000024575 (herein incorporated by reference), registered on 8 Mar. 2017, discusses protein concentrate within the snake fish and its applications as a food supplement for a source of albumin by Professor Dr. Nurpudji Astuti, MPH is also considered. The last patent under consideration is the patent with patent number SID 201808279 (herein incorporated by reference) by LPPM Universitas Muhammadiyah Surakarta about antidiabetic herbal medicine formula containing a combination of snake fish extract and bitter melon extract powder.

Based on the review done on the two patents above, the supplement made of snake fish extract may be packaged mostly in with capsules. The extraction process may be done using a solvent, but the powdering process may be done by boiling, while in this patent, the powdering may be done by drying and milling the solidified extract.

The purpose of this disclosure may be to describe a method for extracting snake fish with high protein content using purified water and ethanol as the solvents. The extract may then be used as a supplement in the form of powder within a capsule, sachet, or a liquid. The production of the snake fish extraction powder may be generally performed via the following steps.
1. Fish selection
2. Cleaning
3. Size reduction
4. Milling
5. Extraction process
6. Drying
7. Powdering
8. Product application

The fish selection may be performed by sorting the fish and selecting those with an average mass of greater or equal to 250 gram, or about 1-4 fishes per kilogram, and the age range of about 9 months old. This specification may be selected to obtain fish with maximum protein content. Generally speaking, the bigger the fish the larger the mass of the flesh, thereby making the protein quantity greater.

The fish cleaning process may be done by removing the scales and guts from the fish as a first step. Gutting may be done to obtain the extraction with no stink and to prevent bacteria contamination during the extraction process. The fish that have been gutted may be washed using flowing water to remove any traces of dirt, blood, other unwanted contaminants within the fish. Gutted and rinsed fish may be considered clean.

The fish that may be already cleaned may then be reduced in size by cutting it into smaller pieces. The cut pieces may have an approximate size between 3 cm to 5 cm from tail to head. The head of the fish may then also be cut into two pieces. The size reduction may be done to ease the milling process.

The cut fish may then be milled using a milling machine. The finer the pulp, the easier the extraction may be. All of the fish parts, including the bones, skin, and head may be milled to obtain increased minerals content, especially calcium with a quantity of 100 mg/500 mg within the extract. The other mineral content that may be contained within the extracts may be Zn: 0.02 mg/500 mg; Fe: 0.03 mg/500 mg; Ca: 1.19 mg/500 mg; Mg: mg/500 mg; and P: 0.63 mg/500 mg.

The milled product may then be extracted using an extraction machine. Ethanol 96% and purified water may be used as the solvent for the milled products. The materials for extraction may then consist of Ethanol 96%, purified water, and milled fish with a composition of 2%-10%, 25%-50%, and 50%-70%, respectively.

Time may be an important parameter during the extraction process. The longer the time, the higher amount of Ethanol may be vaporized, and a higher quantity of extract may be produced. The optimal extraction time to produce the maximum amount of snake fish extract and to vaporize all of the Ethanol may be approximately 24 hours. The extracted liquid produced may be about 40%-50% of the total mass of fish that is used.

Temperature may be an important parameter that affects how long the extraction process may take. The higher the temperature, the quicker the extraction process may be, but such high temperatures may also reduce the overall protein content of the fish. The optimal temperature for the extraction process may be about 50 degrees centigrade to about 65 degrees centigrade. The extraction process may be performed without removing any oil content from the fish, thus producing extract with high omega content. The omega content within the extract may be omega 3, omega 6, and omega 9, each with a concentration of about 10 mg/500 mg, about 10.45 mg/500 mg, and about 4.25 mg/500 mg, respectively.

The extract produced may then be filtered to separate the filtrate (liquid), and the residue (pulp). The filtrate obtained after filtration may be about 40%-50% of the total extract.

The filtrate may then be dried using an oven to solidify it. Oven drying temperatures may range considerably, but typically a range of 50 degrees centigrade to 80 degrees centigrade may be sufficient. Before drying may be initiated, the filtrate may need to be added with Maltodextrin DE 10-12. The concentration of Maltodextrin may constitute approximately 20%-60% and must be dissolved completely within the filtrate to produce a homogeneous solution. The addition of Maltodextrin may be for the general protection of the extract from the heat of the drying process. Such heat from the oven may cause protein breakdown and the addition of Maltodextrin may therefore aid in increasing the dried extract produced.

The dried extract may have a plate-like shape which may be milled with a flour mill to produce the extract powder. This powder may be white but may also have a trace of yellow coloring. The powder may further have a faintly fishy smell. A mesh size of 70-80 may be desired, but not required.

The powder extract may have the highest amino acid content of glutamate and leucine. The quantities of glutamate and leucine may be approximately 67.9 mg/500 mg and 42.45 mg/500 mg respectively but may vary. The contents of pathogen microbes and heavy metals are negative, which means that the extract may be safe to be consumed. The total contents of snake fish extract may be shown in the table below.

Table of snake fish extract contents:

| No | Content | Quantity | Unit | Method |
|---|---|---|---|---|
| 1 | Water content | 2.98 | mg/500 mg | SNI |
| | | | | 01.2892.1992 |
| 2 | Ash content | 12.9 | mg/500 mg | SNI |
| | | | | 01.2892.1992 |
| 3 | Fat content | 5.5 | mg/500 mg | SNI |
| | | | | 01.2892.1992 |
| 4 | Protein content | 394.95 | mg/500 mg | SNI |
| | | | | 01.2892.1992 |
| 5 | Carbohydrate | 5.53 | mg/500 mg | SNI |
| | | | | 01.2892.1992 |
| 6 | Total energy | 370.1 | Kcal/100 g | Calculation |
| 7 | As | Undetected | ppm | SNI |
| | | | | 01.2896.1998 |
| 8 | Cd | Undetected | ppm | SNI |
| | | | | 01.2896.1999 |
| 9 | Hg | Undetected | ppm | SNI |
| | | | | 01.2896.2000 |
| 10 | Pb | Undetected | ppm | SNI |
| | | | | 01.2896.2001 |
| 11 | Zn | 0.02 | mg/500 mg | AAS |
| 12 | Fe | 0.03 | mg/500 mg | AAS |
| 13 | K | 100 | mg/500 mg | AAS |
| 14 | Ca | 1.19 | mg/500 mg | AAS |
| 15 | Mg | 0.19 | mg/500 mg | AAS |
| 16 | P | 0.63 | mg/500 mg | HPLC |
| 17 | Omega 3 | 10 | mg/500 mg | HPLC |
| 18 | Omega 6 | 10.45 | mg/500 mg | HPLC |
| 19 | Omega 9 | 4.25 | mg/500 mg | HPLC |
| 20 | Vitamin A | 0.0045 | mg/500 mg | HPLC |
| 21 | Vitamin B1 | 0.0043 | mg/500 mg | HPLC |
| 22 | Vitamin B6 | 0.003 | mg/500 mg | HPLC |
| 23 | Vitamin B12 | 0.0033 | mg/500 mg | HPLC |
| 24 | Vitamin E | 0.045 | mg/500 mg | HPLC |
| 25 | Vitamin D3 | 0.249 | mg/500 mg | HPLC |
| 26 | Arachidonic Acid | 0.0979 | mg/500 mg | HPLC |
| 27 | Aspartate | 45.55 | mg/500 mg | HPLC |
| 28 | Glycine | 19.8 | mg/500 mg | HPLC |
| 29 | Glutamate | 67.9 | mg/500 mg | HPLC |
| 30 | Lysine | 44.3 | mg/500 mg | HPLC |
| 31 | Tyrosine | 20.5 | mg/500 mg | HPLC |
| 32 | Alanine | 29 | mg/500 mg | HPLC |
| 33 | Leucine | 42.45 | mg/500 mg | HPLC |
| 34 | Valine | 25.15 | mg/500 mg | HPLC |
| 35 | Tryptophan | 15 | mg/500 mg | HPLC |
| 36 | Hydroxyproline | 37.9 | mg/500 mg | HPLC |
| 37 | Prolin | 18 | mg/500 mg | HPLC |
| 38 | Phenylalanine | 23.5 | mg/500 mg | HPLC |
| 39 | Histidine | 14.87 | mg/500 mg | HPLC |
| 40 | Isoleucine | 25.15 | mg/500 mg | HPLC |
| 41 | Threonine | 25.2 | mg/500 mg | HPLC |
| 42 | Methionine | 16.5 | mg/500 mg | HPLC |
| 43 | Serine | 23 | mg/500 mg | HPLC |
| 44 | Sistine | 5.22 | mg/500 mg | HPLC |
| 45 | Total plate count | <1.0 × 10¹ | Colony/g | FI edisi V: |
| | | | | 2014 |
| 46 | Mold yeast count | <1.0 × 10¹ | Colony/g | FI edisi V: |
| | | | | 2015 |
| 47 | Escherichia coli | Negative | /g | FI edisi V: |
| | | | | 2016 |
| 48 | Staphylococcus aureus | Negative | /g | FI edisi V: |
| | | | | 2017 |
| 49 | Pseudomonas | Negative | /g | FI edisi V: |
| | aeruginosa | | | 2018 |
| 50 | Salmonella spp | Negative | /g | FI edisi V: |
| | | | | 2019 |
| 51 | Shigella spp | Negative | /g | ISO |
| | | | | 21567: 2004 |

The powder snake fish extract may be applied as one component of an assortment of ingredients for a supplement formulation. This supplement may be packaged in the form of a capsule, a liquid sachet, and a powder sachet.

The snake fish extract that may be made for a supplement in the form of a capsule may have 100% snake fish extract and a total mass of 500 mg. The main benefit of this supplement may be to increase the albumin content within the body, to prevent and fix hypoalbuminemia conditions along with other medical benefits. This snake fish supplement may be commercialized with any number of brand names.

The snake fish extract that may be made for a supplement in the form of a sachet powder may have 10 grams of mass per sachet. Each sachet may have 40%-50% of snake fish extract, 0.1%-5% Moringa extract, and 0.1%-5% Curcuma extract. The powder may need to be dissolved with water before consumption, the water may be of any temperature with the understanding in the art that hotter water tends to dissolve solids more rapidly. This product may be commercialized with any number of brand names.

Accordingly, a multitude of supplement formulations may be herein described. By way of example, a formulation may include a powder bioextract from a snake fish, from 40% to 50% by weight composition, a Moringa extract, from 0.1% to 5% by weight composition, a Curcuma extract, from 0.1% to 5% by weight composition and, a maltodextrin DE 10-12 filler, from 40% to 59.8% by weight composition. Alternatively, a supplement formulation may also include a powder bioextract from a snake fish, from 40% to 50% by weight composition, a Moringa extract, from 0.1% to 5% by weight composition, a maltodextrin DE 10-12 filler, from 45% to 59.9% by weight composition. Alternatively, a supplement formulation may also include a powder bioextract from a powder bioextract from a snake fish, from 40% to 50% by weight composition, a Curcuma extract, from 0.1% to 5% by weight composition and, a maltodextrin DE 10-12 filler, from 45% to 59.9% by weight composition. These numbers and percentages provided herein are for example only and are not meant to constrain or limit this application in any way.

The snake fish extract that may be made for a supplement in the form of a sachet liquid may be directly consumed. The sachet may have a total volume of 15 ml with 5% snake fish extract combined with Moringa and Curcuma extract. This supplement may be beneficial in the prevention of hypoalbuminemia and a body-wide immunity enhancement. This product may be commercialized with any number of brand names.

While embodiments of the present disclosure have been illustrated and described, it is not intended that these embodiments illustrate and describe all possible forms of this disclosure. Rather, the words used in the specification are words of description rather than limitation, and it is understood that various changes may be made without departing from the spirit and scope of the disclosure.

Accordingly, it is not intended that the various embodiments be limited except by the appended claims. Insofar as the description above and the accompanying drawings disclose any additional subject matter that is not within the scope of the claims below, the embodiments are not dedicated to the public and the right to file one or more applications to claim such additional embodiments is reserved.

What is claimed is:

1. A method of producing a bioextract from a snake fish comprising:
a selecting of a snake fish;
a cleaning of the snake fish;
a sizing reduction of the snake fish;
a milling of the snake fish;
an extracting process of the pulp;
a drying process of a filtrate;
a combining of a maltodextrin DE 10-12 prior to the said drying process;
a powdering of the solidified bioextract; and,
an applying of the filtered bioproduct to a relevant industrial use.

2. The method of producing a bioextract from a snake fish of claim 1, wherein only fish larger than 250 grams and older than 9 months are selected.

3. The method of producing a bioextract from a snake fish of all the preceding claims, wherein a scales and an one or more than one internal organs of the snake fish are removed and the snake fish is rinsed with a freshwater during the cleaning.

4. The method of producing a bioextract from a snake fish of all the preceding claims, wherein the snake fish is cut into smaller pieces than a whole snake fish.

5. The method of producing a bioextract from a snake fish of all the preceding claims, wherein a milling machine is used to mill the snake fish into a pulp, said milling including bones, skin, and head.

6. The method of producing a bioextract from a snake fish of all the preceding claims, wherein the pulp is combined with a purified water and a concentration of about 96% ethanol to produce an extraction medium.

7. The method of producing a bioextract from a snake fish of all the preceding claims, wherein the extraction of the extraction medium proceeds by use of an extraction machine.

8. The method of producing a bioextract from a snake fish of all the preceding claims, wherein the ratio of the extraction medium is 2% to 10% of 96% Ethanol, 25% to 50% of purified water, and 50% to 70% snake fish pulp.

9. The method of producing a bioextract from a snake fish of all the preceding claims, the extracting of the extraction medium occurring over about 24 hours; said extracting process being complete when the 96% Ethanol has evaporated.

10. The method of producing a bioextract from a snake fish of all the preceding claims, wherein no naturally occurring oils from the snake fish are removed during the extraction process.

11. The method of producing a bioextract from a snake fish of all the preceding claims, wherein the extracting of the extraction medium occurring at a temperature of about 50 degrees centigrade to about 65 degrees centigrade.

12. The method of producing a bioextract from a snake fish of all the preceding claims, wherein the completed extracting process leaves behind an extracted medium.

13. The method of producing a bioextract from a snake fish of all the preceding claims, wherein the extracted medium is filtered to separate a filtrate from a residue, said filtrate being about 40% to 50% of the extracted medium.

14. The method of producing a bioextract from a snake fish of all the preceding claims, wherein the filtrate is dried in an oven with a temperature of about 50 to 80 degrees centigrade.

15. The method of producing a bioextract from a snake fish of all the preceding claims, wherein the maltodextrin DE 10-12 is between 20% to 60% weight concentrations.

16. The method of producing a bioextract from a snake fish of all the preceding claims, wherein the powdering of the solidified bioextract occurs by the use of the milling machine to produce a powdered bioextract.

17. The method of producing a bioextract from a snake fish of all the preceding claims, wherein the powdered bioextract is packaged in the form of a capsule, a liquid sachet, or a powdered sachet.

18. The method of producing a bioextract from a snake fish of all the preceding claims, wherein the powdered bioextract is combined with Moringa extract, Curcuma extract, or Moringa extract and Curcuma extract.

19. A method of producing a bioextract from a snake fish comprising:
a selecting of a snake fish wherein only fish larger than 250 grams and older than 9 months are selected;
a cleaning of the snake fish wherein a scales and an one or more than one internal organs of the snake fish are removed and the snake fish is rinsed with a fresh water during the cleaning;
a sizing reduction of the snake fish wherein the snake fish is cut into smaller pieces than a whole snake fish;
a milling of the snake fish wherein a milling machine is used to mill the snake fish into a pulp, said milling including bones, skin, and head;
an extracting process of the pulp wherein the pulp is combined with a purified water and a concentration of about 96% ethanol to produce an extraction medium;
a drying process of a filtrate wherein the filtrate is dried in an oven with a temperature of about 50 to 80 degrees centigrade;
a combining of a maltodextrin DE 10-12 prior to the said drying process wherein the maltodextrin DE 10-12 is between 20% to 60% weight concentrations;
a powdering of the solidified bioextract wherein the powdering of the solidified bioextract occurs by the use of the milling machine to produce a powdered bioextract; and,
an applying of the filtered bioproduct to a relevant industrial use.

20. The method of producing a bioextract from a snake fish of claim 19, wherein the extraction of the extraction medium proceeds by use of an extraction machine.

21. The method of producing a bioextract from a snake fish of all the preceding claims, wherein the ratio of the extraction medium is 2% to 10% of 96% Ethanol, 25% to 50% of purified water, and 50% to 70% snake fish pulp.

22. The method of producing a bioextract from a snake fish of all the preceding claims, the extracting of the extraction medium occurring over about 24 hours; said extracting process being complete when the 96% Ethanol has evaporated.

23. The method of producing a bioextract from a snake fish of all the preceding claims, wherein no naturally occurring oils from the snake fish are removed during the extraction process.

24. The method of producing a bioextract from a snake fish of all the preceding claims, wherein the extracting of the extraction medium occurring at a temperature of about 50 degrees centigrade to about 65 degrees centigrade.

25. The method of producing a bioextract from a snake fish of all the preceding claims, wherein the completed extracting process leaves behind an extracted medium.

26. The method of producing a bioextract from a snake fish of all the preceding claims, wherein the extracted medium is filtered to separate a filtrate from a residue, said filtrate being about 40% to 50% of the extracted medium.

27. The method of producing a bioextract from a snake fish of all the preceding claims, wherein the powdered bioextract is packaged in the form of a capsule, a liquid sachet, or a powdered sachet.

28. The method of producing a bioextract from a snake fish of all the preceding claims, wherein the powdered bioextract is combined with Moringa extract, Curcuma extract, or Moringa extract and Curcuma extract.

29. A method of producing a bioextract from a species of snake fish, Channa striata, comprising:
a selecting of a snake fish species Channa striata;
a cleaning of the snake fish;
a sizing reduction of the snake fish;
a milling of the snake fish;
an extracting process of the pulp;
a drying process of a filtrate;
a combining of a maltodextrin DE 10-12 prior to the said drying process;
a powdering of the solidified bioextract; and,
an applying of the filtered bioproduct to a relevant industrial use.

30. The method of producing a bioextract from a species of snake fish, Channa striata, of claim 29, wherein only fish larger than 250 grams and older than 9 months are selected.

31. The method of producing a bioextract from a species of snake fish, Channa striata, of all the preceding claims, wherein a scales and an one or more than one internal organs of the snake fish are removed and the snake fish is rinsed with a freshwater during the cleaning.

32. The method of producing a bioextract from a species of snake fish, Channa striata, of all the preceding claims, wherein the snake fish is cut into smaller pieces than a whole snake fish.

33. The method of producing a bioextract from a species of snake fish, Channa striata, of all the preceding claims, wherein a milling machine is used to mill the snake fish into a pulp, said milling including bones, skin, and head.

34. The method of producing a bioextract from a species of snake fish, Channa striata, of all the preceding claims, wherein the pulp is combined with a purified water and a concentration of about 96% ethanol to produce an extraction medium.

35. The method of producing a bioextract from a species of snake fish, Channa striata, of all the preceding claims, wherein the extraction of the extraction medium proceeds by use of an extraction machine.

36. The method of producing a bioextract from a species of snake fish, Channa striata, of all the preceding claims, wherein the ratio of the extraction medium is 2% to 10% of 96% Ethanol, 25% to 50% of purified water, and 50% to 70% snake fish pulp.

37. The method of producing a bioextract from a species of snake fish, Channa striata, of all the preceding claims, the extracting of the extraction medium occurring over about 24 hours; said extracting process being complete when the 96% Ethanol has evaporated.

38. The method of producing a bioextract from a species of snake fish, Channa striata, of all the preceding claims, wherein no naturally occurring oils from the snake fish are removed during the extraction process.

39. The method of producing a bioextract from a species of snake fish, Channa striata, of all the preceding claims, wherein the extracting of the extraction medium occurring at a temperature of about 50 degrees centigrade to about 65 degrees centigrade.

40. The method of producing a bioextract from a species of snake fish, Channa striata, of all the preceding claims, wherein the completed extracting process leaves behind an extracted medium.

41. The method of producing a bioextract from a species of snake fish, Channa striata, of all the preceding claims, wherein the extracted medium is filtered to separate a filtrate from a residue, said filtrate being about 40% to 50% of the extracted medium.

42. The method of producing a bioextract from a species of snake fish, Channa striata, of all the preceding claims, wherein the filtrate is dried in an oven with a temperature of about 50 to 80 degrees centigrade.

43. The method of producing a bioextract from a species of snake fish, Channa striata, of all the preceding claims, wherein the maltodextrin DE 10-12 is between 20% to 60% weight concentrations.

44. The method of producing a bioextract from a species of snake fish, Channa striata, of all the preceding claims, wherein the powdering of the solidified bioextract occurs by the use of the milling machine to produce a powdered bioextract.

45. The method of producing a bioextract from a snake fish of all the preceding claims, wherein the powdered bioextract is packaged in the form of a capsule, a liquid sachet, or a powdered sachet.

46. The method of producing a bioextract from a species of snake fish, Channa striata, of all the preceding claims, wherein the powdered bioextract is combined with Moringa extract, Curcuma extract, or Moringa extract and Curcuma extract.

47. An extraction process of a snake fish comprising:
a combining of a snake fish pulp with a purified water and an ethanol, the ethanol having a concentration of about 96%, said combination making an extraction medium;
a placing of the extraction medium into an extraction machine;
a waiting for the extraction of the extraction medium to occurring over about 24 hours;
a temperature controlling of the extraction process wherein the extraction medium is extracted at a temperature of about 50 degrees centigrade to about 65 degrees centigrade, said extracting process being complete when the 96% ethanol has evaporated leaving behind an extracted medium;
a filtration of the extracted medium wherein a filter paper is used to separate a filtrate from a residue, said filtrate being about 40% to 50% of the extracted medium;
a drying of the filtrate to make a solid bioextract;
a milling of the solid bioextract to make a powered bioextract.

48. The extraction process of a snake fish of claim 47, wherein the ratio of the extraction medium is 2% to 10% of 96% Ethanol, 25% to 50% of purified water, and 50% to 70% snake fish pulp.

49. The extraction process of a snake fish of all the proceeding claims, wherein no naturally occurring oils from the snake fish are removed during the extraction process.

50. The extraction process of a snake fish of all the proceeding claims, wherein a maltodextrin DE 10-12 is added before the drying process and is between 20% to 60% weight concentration.

51. The extraction process of a snake fish of all the proceeding claims, wherein the milling of the solid bioextract occurs by the use of the milling machine to produce a powdered bioextract.

52. The extraction process of a snake fish of all the proceeding claims, wherein the powdered bioextract is packaged in the form of a capsule, a liquid sachet, or a powdered sachet.

53. The extraction process of a snake fish of all the proceeding claims, wherein the powdered bioextract is combined with Moringa extract, Curcuma extract, or Moringa extract and Curcuma extract.

54. An extraction process of a species of snake fish, Channa striata, comprising:
a combining of a snake fish pulp, derived from the species Channa striata, with a purified water and an ethanol, said ethanol having a concentration of about 96%, said combination making an extraction medium;
a placing of the extraction medium into an extraction machine;
a waiting for the extraction of the extraction medium to occurring over about 24 hours;
a temperature controlling of the extraction process wherein the extraction medium is extracted at a temperature of about 50 degrees centigrade to about 65 degrees centigrade, said extracting process being complete when the 96% ethanol has evaporated leaving behind an extracted medium;
a filtration of the extracted medium wherein a filter paper is used to separate a filtrate from a residue, said filtrate being about 40% to 50% of the extracted medium;
a drying of the filtrate to make a solid bioextract;
a milling of the solid bioextract to make a powered bioextract.

55. The extraction process of a species of snake fish, Channa striata, of claim 54, wherein the ratio of the extraction medium is 2% to 10% of 96% Ethanol, 25% to 50% of purified water, and 50% to 70% snake fish pulp.

56. The extraction process of a species of snake fish, Channa striata, of all the proceeding claims, wherein no naturally occurring oils from the snake fish are removed during the extraction process.

57. The extraction process of a species of snake fish, Channa striata, of all the proceeding claims, wherein a maltodextrin DE 10-12 is added before the drying process and is between 20% to 60% weight concentration.

58. The extraction process of a species of snake fish, Channa striata, of all the proceeding claims, wherein the milling of the solid bioextract occurs by the use of the milling machine to produce a powdered bioextract.

59. The extraction process of a species of snake fish, Channa striata, of all the proceeding claims, wherein the powdered bioextract is packaged in the form of a capsule, a liquid sachet, or a powdered sachet.

60. The extraction process of a species of snake fish, Channa striata, of all the proceeding claims, wherein the powdered bioextract is combined with Moringa extract, Curcuma extract, or Moringa extract and Curcuma extract.

61. A supplement formulation, comprising:
(a) a powder bioextract from a snake fish, from 40% to 50% by weight composition;
(b) a Moringa extract, from 0.1% to 5% by weight composition;
(c) a Curcuma extract, from 0.1% to 5% by weight composition;
(d) a maltodextrin DE 10-12 filler, from 40% to 59.8% by weight composition.

62. A supplement formulation, comprising:
(a) a powder bioextract from a snake fish, from 40% to 50% by weight composition;
(b) a Moringa extract, from 0.1% to 5% by weight composition;
(c) a maltodextrin DE 10-12 filler, from 45% to 59.9% by weight composition.

63. A supplement formulation, comprising:
(a) a powder bioextract from a snake fish, from 40% to 50% by weight composition;
(b) a Curcuma extract, from 0.1% to 5% by weight composition;
(c) a maltodextrin DE 10-12 filler, from 45% to 59.9% by weight composition.

64. A supplement formulation derived from the species Channa striata, comprising:
(a) a powder bioextract from a snake fish species Channa striata, from 40% to 50% by weight composition;
(b) a Moringa extract, from 0.1% to 5% by weight composition;
(c) a Curcuma extract, from 0.1% to 5% by weight composition;
(d) a maltodextrin DE 10-12 filler, from 40% to 59.8% by weight composition.

65. A supplement formulation derived from the species Channa striata, comprising:
(a) a powder bioextract from a snake fish species Channa striata, from 40% to 50% by weight composition;
(b) a Moringa extract, from 0.1% to 5% by weight composition;
(c) a maltodextrin DE 10-12 filler, from 45% to 59.9% by weight composition.

66. A supplement formulation derived from the species Channa striata, comprising:
(a) a powder bioextract from a snake fish species Channa striata, from 40% to 50% by weight composition;
(b) a Curcuma extract, from 0.1% to 5% by weight composition;
(c) a maltodextrin DE 10-12 filler, from 45% to 59.9% by weight composition.
